(19) United States
(12) Patent Application Publication
(10) Pub. No.: US 2024/0343670 A1
(43) Pub. Date: Oct. 17, 2024

(54) PROCESS FOR PRODUCING RECYCLED LACTATE SALT

(71) Applicant: TRIPLEW LTD., Netanya (IL)

(72) Inventors: Tal Shapira, Even Yehuda (IL); Nitsan Papo, Kefar Sava (IL); Dina Pinsky, Jerusalem (IL)

(21) Appl. No.: 18/293,759

(22) PCT Filed: Aug. 2, 2022
(86) PCT No.: PCT/IL2022/050832

§ 371 Date: Jan. 30, 2024

Related U.S. Application Data
(60) Provisional application No. 63/314,416, filed on Feb. 27, 2022.
(60) Provisional application No. 63/228,618, filed on Aug. 3, 2021.

(51) Int. Cl. C07C 51/41; C07C 51/43; C12P 7/56

(52) U.S. Cl. CPC C07C 51/412; C07C 51/43; C12P 7/56

(57) ABSTRACT

The present invention relates to processes for producing highly pure magnesium L-lactate salt from decomposed organic waste and enriching the enantiomeric purity of recycled lactate salt.

### FIELD OF THE INVENTION

The present invention relates to a process for producing magnesium L-lactate in high enantiomeric purity and low amounts of non-lactate impurities. The present invention further relates to a process for enriching the enantiomeric purity of recycled lactate salt containing an enantiomeric mixture of L- and D-lactate.

### BACKGROUND OF THE INVENTION

### Lactic Acid Fermentation

Lactic acid fermentation, namely, production of lactic acid from carbohydrate sources via microbial fermentation, has been gaining interest in recent years due to the ability to use lactic acid as a building block in the manufacture of bioplastics. Lactic acid can be polymerized to form the biodegradable and recyclable polyester, polylactic acid (PLA), which is considered a potential substitute for plastics manufactured from petroleum. PLA is used in the manufacture of various products including food packaging, disposables, fibers in the textile and hygiene products industries, and more.

Production of lactic acid by fermentation bioprocesses is preferred over chemical synthesis methods for various considerations, including environmental concerns, costs and the difficulty to generate enantiomerically pure lactic acid by chemical synthesis, which is desired for most industrial applications. The conventional fermentation process is typically based on anaerobic fermentation by lactic acid-producing microorganisms, which produce lactic acid as the major metabolic end product of carbohydrate fermentation. For production of PLA, the lactic acid generated during the fermentation is separated from the fermentation broth and purified by various processes, and the purified lactic acid is then subjected to polymerization.

Lactic acid has a chiral carbon atom and therefore exists in two enantiomeric forms, D- and L-lactic acid. In order to generate PLA that is suitable for industrial applications, the D- or L-lactic acid entering the production process must be highly purified to meet the specification required for polymerization and reuse. Lactic acid bacteria that produce only L-lactate enantiomer or only D-lactate enantiomer are typically used in order to produce one discreet enantiomer (L or D, respectively).

In currently available commercial processes, the carbohydrate source for lactic acid fermentation is typically a starch-containing renewable source such as corn and cassava root. Additional sources, such as the cellulose-rich sugarcane bagasse, have also been proposed. Typically, lactic acid-producing bacteria can utilize reducing sugars such as glucose and fructose, but do not have the ability to degrade polysaccharides such as starch and cellulose. Thus, to utilize such polysaccharides, the process requires adding glycolytic enzymes, typically in combination with chemical treatment, to degrade the polysaccharides and release reducing sugars.

An additional source of carbohydrates for lactic acid fermentation that has been proposed is complex organic waste, such as mixed food waste from municipal, industrial and commercial origin. Organic waste is advantageous as it is readily available and less expensive compared to other carbohydrate sources for lactic acid fermentation.

Mixed food waste typically includes varied ratios of reducing sugars (glucose, fructose, lactose, etc.), starch and lignocellulosic material. Mixed food waste also contains endogenous D,L-lactic acid (e.g., from dairy products or natural decomposition during transportation), one of which needs to be removed in order to utilize the waste as a substrate for producing optically pure lactic acid (L- or D-lactic acid). WO 2017/122197, assigned to the Applicant of the present invention, discloses dual action lactic-acid (LA)-utilizing bacteria genetically modified to secrete polysaccharide-degrading enzymes such as cellulases, hemicellulases, and amylases, useful for processing organic waste both to eliminate lactic acid present in the waste and degrade complex polysaccharides. WO 2020/208635, assigned to the Applicant of the present invention, discloses systems and methods for processing organic waste, particularly mixed food waste, using a D-lactate oxidase, which eliminates D-lactic acid that is present in the organic waste.

### Polylactic Acid (PLA) Recycling

PLA produced from renewable resources is an alternative to petroleum-derived plastics, and its use in the manufacture of products such as food packaging is continuously growing. Due to the increasing presence of PLA in disposable end products, it is important to ensure that PLA is adequately addressed after disposal. Unlike thermoplastic resins such as polyethylene, polypropylene, polystyrene and poly(ethylene terephthalate), PLA is subject to thermal degradation. Accordingly, when products containing a mixture of PLA and the aforementioned plastics are recycled, it is desirable to separate PLA in order to avoid contamination of the recycling streams.

Recycling options for PLA include landfilling, composting, anaerobic digestion (biogas production), incineration and chemical recycling into the constituent monomers. Chemical recycling is preferred over other methods as the monomers can be reused in the production of new PLA.

One of the common forms of PLA on the market is the copolymer PDLLA (poly(D-L-) lactic acid), predominantly composed of PLLA (made from L-lactic acid), and small amounts of PDLA (made from D-lactic acid). A significant portion of the PLA plastics present on the market contains a small amount of PDLA that when hydrolyzed, releases D-lactic acid. The hydrolyzed material may also contain unknown amounts of D-lactic acid formed by racemization during the hydrolysis. An optical purity of over 99% is typically required for both D-lactic acid and L-lactic acid entering the PLA production process. Therefore, PLA recycling processes should address the issue of isomer separation. Chemical separation of the two enantiomers is expensive, usually using liquid or solid enantioselective membranes or high-performance liquid chromatography (HPLC).

Cam, Hyon and Ikada (1995) Biomaterials, 16 (11): 833-43, report the degradation of high molecular weight poly(L-lactide) in alkaline medium. The study tested the effect of molecular weight and morphology on hydrolytic degradation. Degradation was performed at 37° C. in 0.01 N NaOH solution.

Siparsky, Voorhees, and Miao (1998) Journal of environmental polymer degradation, 6 (1): 31-41, report the hydrolysis of polylactic acid (PLA) and polycaprolactone (PCL) in aqueous acetonitrile solutions.

Xu, Crawford and Gorman (2011) Macromolecules, 44 (12): 4777-4782, report the effects of temperature and pH on the degradation of poly(lactic acid) brushes.

Chauliac (2013) “Development of a thermochemical process for hydrolysis of polylactic acid polymers to L-lactic acid and its purification using an engineered microbe” Ph.D. thesis, University of Florida, UMI Number: 3583516, proposes a process for post-consumer use of PLA polymers. In this process, thermohydrolysis is the first step, followed by D-LA removal from the hydrolyzed material to yield pure L-LA that could be redirected into the production of the polymer itself. Thermohydrolysis was performed with water in the presence of NaOH. D-LA removal from the resulting syrup was achieved using an Escherichia coli lacking all three L-lactate dehydrogenases identified.

Wadsö and Karlsson (2013) Polymer Degradation and Stability, 98 (1): 73-78, report two studies to measure the enthalpy of alkaline hydrolysis of polymers containing esters of carboxylic acids. Two materials were used: poly(vinyl acetate), PVAc, films and poly(lactic acid), PLA, fibers. Degradation was carried out using sodium hydroxide and potassium hydroxide at 30° C.

Elsawy et al. (2017) Renewable and Sustainable Energy Reviews, 79:1346-1352, review the hydrolytic degradation of polylactic acid (PLA) and its composites.

Motoyama et al. (2007) Polymer Degradation and Stability, 92 (7): 1350-1358, report the effects of MgO catalyst on depolymerization of poly-L-lactic acid to L,L-lactide.

WO 2015/112098 discloses a process for manufacturing lactide from plastics having polylactic acid (PLA-based plastics) that comprises preparing PLA-based plastics, accelerating decomposition of polylactic acid in the plastics by alcoholysis or hydrolysis to provide low molecular weight polylactic acid, and thermal decomposition of the low molecular weight polylactic acid to provide lactide. Also, the process further comprises minimizing the size of the PLA-based plastics after the preparation step, and purifying lactide after thermal decomposition of the low molecular weight polylactic acid.

U.S. Pat. No. 7,985,778 discloses a method for decomposing and reclaiming synthetic resin having ester bond in composition structure thereof, by conducting hydrolysis treatment and then separation collection treatment. In the hydrolysis treatment, an article containing synthetic resin to be decomposed and reclaimed is exposed to water vapor atmosphere filled under saturation water vapor pressure at treatment temperature at or below melting point of the synthetic resin. The synthetic resin in article to be treated is hydrolyzed by water vapor generated at the treatment temperature, to generate decomposition product before polymerizing to the synthetic resin containing an ester bond. The separation collection treatment is treatment in which the decomposition product generated by the hydrolysis treatment is separated into liquid component and solid component to be collected individually.

U.S. Pat. No. 8,614,338 discloses a method for the stereospecific chemical recycling of a mixture of polymers based on polylactic acid PLA, in order to reform the monomer thereof or one of the derivatives thereof. The method comprises a step of putting the mixture of polymers in suspension in a lactic ester able to dissolve the PLA fraction followed by a separation firstly of the lactic ester, the PLA and other dissolved impurities and secondly the mixture of other polymers and impurities that are insoluble. The solution containing the PLA thus obtained is then subjected to a catalytic depolymerization reaction by transesterification in order to form oligoesters. The depolymerization reaction by transesterification is then stopped at a given moment and the residual lactic ester separated. The oligoester thus obtained then undergoes a cyclisation reaction in order to produce lactide that will finally be purified stereospecifically so as to obtain a fraction of purified lactide having a meso-lactide content of between 0.1% and 40%.

U.S. Pat. Nos. 8,431,683 and 8,481,675 disclose a process for recycling a polymer blend necessarily containing PLA, comprising grinding, compacting, dissolving in a solvent of PLA, removing the undissolved contaminating polymers, alcoholysis depolymerization reaction and purification steps.

U.S. Pat. No. 8,895,778 discloses depolymerization of polyesters such as post-consumer polylactic acid. Ultrasonic induced implosions can be used to facilitate the depolymerization. Post-consumer PLA was exposed to methanol as the suspension media in the presence of organic or ionic salts of alkali metals such a potassium carbonate and sodium hydroxide as depolymerization catalysts to provide high quality lactic acid monomers in high yield.

U.S. 2018/0051156 discloses a method for enhancing/accelerating the depolymerization of polymers (e.g., those containing hydrolyzable linkages), the method generally involves contacting a polymer comprising hydrolyzable linkages with a solvent and an alcohol to give a polymer mixture in which the polymer is substantially dissolved, wherein the contacting is conducted at a temperature at or below the boiling point of the polymer mixture. A resulting depolymerized polymer can be separated therefrom (including, e.g., monomers and/or oligomers). Such methods can be conducted under relatively mild temperature and pressure conditions. In some embodiments, the polymer is poly(lactic acid).

WO 2021/165964, assigned to the Applicant of the present invention, discloses industrial fermentation for the production of lactic acid from organic waste combined with chemical recycling of polylactic acid to obtain lactic acid at high yields.

There remains an unmet need for economical and reliable processes for generation of enantiomerically pure L-lactate salt from decomposed waste containing both L- and D-lactate enantiomers.

### SUMMARY OF THE INVENTION

The present invention provides highly pure L-lactate monomers from lactic acid fermentation of organic waste and/or chemical hydrolysis of PLA. The present invention also provides a process for enriching the enantiomeric purity of lactate salt obtained from recycling of organic waste and/or PLA waste.

The present invention is based, in part, on the unexpected finding that the enantiomeric purity of L-lactate can be increased by performing ion exchange or swap of the lactate counterion in a fermentation broth or a PLA hydrolysis slurry containing various concentrations of D-lactate monomers to result in the enantioselective precipitation of L-lactate salt, specifically magnesium L-lactate salt. The present invention therefore enables the recycling of waste from various sources including waste that contains endogenous D-lactate monomers while obviating the need for D-lactic acid-utilizing bacteria or enzymes to eliminate D-lactate monomers. The present invention advantageously produces L-lactate in enantiomeric purity of over 99% which can be used for the commercial production of PLA with no additional processing for isomer separation.

According to the principles of the present invention, both the fermentation of organic waste and the hydrolysis of PLA are performed in the presence of an alkaline compound. During fermentation, the pH in the fermenter decreases due to the production of lactic acid, which adversely affects the productivity of the lactic acid-producing microorganism. Thus, an alkaline compound, typically sodium, potassium, ammonium or magnesium hydroxide, and a mixture or combination thereof, is added to neutralize the pH thereby resulting in the formation of a lactate salt. The present invention discloses for the first time the use of ammonia water (aqua ammonia) derived from anaerobic digestion of a solid biomass waste of a fermentation process as the source of alkaline compound during fermentation or PLA hydrolysis. Ammonia water derived from a solid biomass of a previous fermentation process is an excellent source of alkalinity to be added to adjust the pH of the fermentation broth to a desired value while also affording significant cost savings by obviating the need for costly alkaline compound to be added. Furthermore, it provides an additional recycling of the solid biomass that is obtained after lactic acid fermentation.

Additional advantages of the process of the invention stem from using recycled salt from previous acidification of a lactate salt to afford the ion exchange or swap of the lactate counterion in a fermentation broth or a PLA hydrolysis slurry. Typically, both lactic acid fermentation processes and PLA decomposition processes result in a lactate salt. In order to obtain polylactic acid, the lactate salt needs to be acidified to lactic acid monomers or methylated or acetylated in the presence of an acid. Thus, an acid such as hydrochloric acid, hydrobromic acid, nitric acid, phosphoric acid, sulfuric acid, and a mixture or combination thereof is used. During these processes, the counterion of the lactate salt and anion of the acid precipitate to form a salt. According to the principles of the present invention, this salt can be reused in a subsequent process of fermentation or PLA hydrolysis as a source of ions for ion exchange or swap of the lactate counterion.

According to a first aspect, a process for enriching L-lactate enantiomer from an enantiomeric mixture derived from decomposed organic waste is provided, the process comprising the steps of:
(a) obtaining decomposed organic waste comprising an enantiomeric mixture of D- and L-lactate and a counterion other than magnesium;
(b) optionally performing at least one of neutralizing the D- and L-lactate and removing solid particles from the decomposed organic waste; and
(c) adding a magnesium salt to the enantiomeric mixture of step (a) or (b) to thereby precipitate magnesium L-lactate salt with enriched enantiomeric purity.

In one embodiment, the process provides enrichment of L-lactate enantiomer by 1% or more. In another embodiment, the process provides enrichment of L-lactate enantiomer by 5% or more. In yet another embodiment, the process provides enrichment of L-lactate enantiomer by 10% or more. In particular embodiments, the process provides enrichment of L-lactate enantiomer of up to 15%. In other embodiments, the process provides enrichment of L-lactate enantiomer of up to 20%. In yet other embodiments, the process provides enrichment of L-lactate enantiomer of up to 25%.

In certain embodiments, the decomposed organic waste is obtained from a lactic acid fermentation process. In further embodiments, the decomposed organic waste is obtained from a lactic acid-containing waste. In additional embodiments, the decomposed organic waste is obtained from hydrolysis of polylactic acid polymer.

In various embodiments, the organic waste comprises a carbohydrate source. In other embodiments, the organic waste is selected from food waste, municipal food waste, residential food waste, agricultural waste, industrial food waste from food processing facilities, commercial food waste (from hospitals, restaurants, shopping centers, airports etc.), and a mixture or combination thereof. Each possibility represents a separate embodiment.

In further embodiments, the decomposed organic waste is pre-treated prior to step (a). In particular embodiments, pretreatment comprises removal of non-lactic acid-containing impurities.

According to the principles of the present invention, the decomposed organic waste contains endogenous D-lactate in an amount of up to 20 wt. %. In some embodiments, the enantiomeric mixture comprises 20% D-lactate or less. In other embodiments, the enantiomeric mixture comprises 10% D-lactate or less. In yet other embodiments, the enantiomeric mixture comprises 5% D-lactate or less.

In certain embodiments, the counterion is selected from the group consisting of sodium, potassium and ammonium. Each possibility represents a separate embodiment. While the process of the present invention utilizes decomposed organic waste comprising an enantiomeric mixture of D- and L-lactate and a counterion other than magnesium, it is contemplated that magnesium ions can be present in the decomposed organic waste. Thus, according to various embodiments, the decomposed organic waste comprises an enantiomeric mixture of D- and L-lactate, magnesium ions, and a counterion other than magnesium.

In further embodiments, step (b) comprising neutralizing the D- and L-lactate is performed. Typically, neutralization is performed to a pH of about 6.5 to about 7.5, including each value within the specified range. In one embodiment, neutralization is performed in the presence of an acid selected from hydrochloric acid, hydrobromic acid, nitric acid, phosphoric acid, sulfuric acid, and combinations thereof. Each possibility represents a separate embodiment. In one embodiment, neutralization is performed in the presence of sulfuric acid. In other embodiments, neutralization is performed in the presence of a base selected from sodium, potassium, or ammonium hydroxide, and combinations thereof. Each possibility represents a separate embodiment.

In various embodiments, step (b) comprising removing solid particles from the decomposed organic waste is performed and removal of solid particles comprises solid-liquid separation.

In additional embodiments, step (c) is performed at elevated temperatures. In some embodiments, step (c) is performed at temperatures in the range of 20° C. to 80° C., including each value within the specified range.

In certain embodiments, the magnesium salt in step (c) is added in solid form. In alternative embodiments, the magnesium salt in step (c) is added as an aqueous solution. In further embodiments, the magnesium salt in step (c) is gradually added. In other embodiments, the magnesium salt in step (c) is added in excess of up to 20%. In further embodiments, the magnesium salt in step (c) is derived from acidification, methylation or acetylation of magnesium L-lactate of a previous batch.

In particular embodiments, the magnesium salt in step (c) is magnesium sulfate.

In additional embodiments, the obtained magnesium L-lactate salt is separated by filtration or centrifugation. In further embodiments, the obtained magnesium L-lactate salt is subjected to subsequent purification. In other embodiments, subsequent purification comprises at least one of crystallization, recrystallization, partitioning, silica gel chromatography, preparative HPLC, and combinations thereof. Each possibility represents a separate embodiment.

In particular embodiments, subsequent purification comprises washing the obtained magnesium L-lactate salt, for example using purified water. In other embodiments, subsequent purification comprises dissolving and recrystallizing the obtained magnesium L-lactate salt.

In one embodiment, the obtained magnesium L-lactate salt comprises less than 3% magnesium D-lactate. In another embodiment, the obtained magnesium L-lactate salt comprises less than 2% magnesium D-lactate. In yet another embodiment, the obtained magnesium L-lactate salt comprises less than 1.5% magnesium D-lactate. In specific embodiments, the obtained magnesium L-lactate salt comprises less than 1% magnesium D-lactate.

In further embodiments, the obtained magnesium L-lactate salt is crystalline magnesium L-lactate dihydrate.

In other embodiments, the obtained magnesium L-lactate is acidified to form L-lactic acid by at least one of hydrochloric acid, hydrobromic acid, nitric acid, phosphoric acid, sulfuric acid, and combinations thereof. Each possibility represents a separate embodiment. In particular embodiments, the L-lactic acid is used for subsequent polylactic acid formation.

In some embodiments, the process disclosed herein further comprises enriching the purity of L-lactate salt from decomposed organic waste.

According to another aspect, a process for enriching the purity of L-lactate salt from decomposed organic waste is provided, the process comprising the steps of:
(a) obtaining decomposed organic waste comprising L-lactate and a counterion other than magnesium;
(b) optionally performing at least one of neutralizing the L-lactate and removing solid particles from the decomposed organic waste; and
(c) adding a magnesium salt to the decomposed organic waste of step (a) or (b) to thereby precipitate magnesium L-lactate salt with enriched purity.

According to yet another aspect, a process for producing magnesium L-lactate salt from decomposed organic waste in high purity is provided, the process comprising the steps of:
(a) obtaining decomposed organic waste comprising L-lactate and a counterion other than magnesium;
(b) optionally performing at least one of neutralizing the L-lactate and removing solid particles from the decomposed organic waste; and
(c) adding a magnesium salt to the decomposed organic waste of step (a) or (b) to thereby precipitate magnesium L-lactate salt in high purity.

According to a further aspect, a process for producing magnesium L-lactate salt from decomposed organic waste is provided, the process comprising the steps of:
(a) decomposing organic waste by performing at least one of organic waste fermentation using a lactic acid-producing microorganism and PLA hydrolysis in the presence of an alkaline compound to obtain decomposed organic waste comprising L-lactate and a counterion other than magnesium;
(b) optionally performing at least one of neutralizing the L-lactate and removing solid particles from the decomposed organic waste; and
(c) adding a magnesium salt to the decomposed organic waste of step (a) or (b) to thereby precipitate magnesium L-lactate salt.

In some embodiments, the alkaline compound comprises at least one of NaOH, KOH, NH₄OH, Ca(OH)₂, and a mixture or combination thereof. Each possibility represents a separate embodiment. In other embodiments, the alkaline compound comprises a combination of Mg(OH)₂ and/or MgCO₃ and at least one of NaOH, KOH, NH₄OH, Ca(OH)₂, and a mixture or combination thereof. In further embodiments, the alkaline compound comprises NH₄OH derived from anaerobic digestion of a solid biomass obtained from a previous batch of lactic acid fermentation. In various embodiments, the NH₄OH derived from anaerobic digestion of a solid biomass is obtained by gas stripping.

In some embodiments, the magnesium salt in step (c) is derived from acidification, methylation or acetylation of magnesium L-lactate of a previous batch of lactic acid fermentation. In other embodiments, the magnesium salt in step (c) is derived from acidification, methylation or acetylation of magnesium L-lactate of a previous batch of PLA hydrolysis.

Other objects, features and advantages of the present invention will become clear from the following description, examples and drawings.

### BRIEF DESCRIPTION OF THE FIGURES

FIG. 1. Schematic representation of a process according to certain embodiments of the present invention.

FIG. 2. Lactate concentration (●) and % D-lactate (x) concentration in a solution during a swap reaction of sodium lactate (NaLa) produced from hydrolyzing PLA grade number 4032D according to certain embodiments of the present invention.

### DETAILED DESCRIPTION OF THE INVENTION

The present invention provides a process for producing magnesium L-lactate salt from decomposed organic waste in high enantiomeric purity and reduced amounts of impurities. The present invention further provides processes for enriching L-lactate enantiomer from an enantiomeric mixture of D- and L-lactate and enriching the purity of L-lactate salt from decomposed organic waste. The high purity magnesium L-lactate salt can further be used for generating new lactic acid-based products.

As used herein, the term “lactic acid” refers to the hydroxycarboxylic acid having the following chemical formula CH₃CH(OH)CO₂H. The terms lactic acid or lactate (unprotonated lactic acid) can refer to the stereoisomers (enantiomers) of lactic acid: L-lactic acid/L-lactate, D-lactic acid/D-lactate, or to a combination thereof. The term “enantiomers” as used herein refers to two stereoisomers of a compound which are non-superimposable mirror images of one another.

For most industrial applications, L-lactic acid monomers with high purity are required in order to produce PLA with suitable properties. Thus, the processes of the present invention are directed, in particular, to the production of L-lactate salts with enriched enantiomeric purity or chiral purity, which can be converted to L-lactic acid suitable for reuse without the necessity to eliminate D-lactate monomers.

One advantage stemming from the processes of the present invention is enantiomeric enrichment, which is particularly beneficial for reuse of lactic acid. The enrichment of L-lactate enantiomer by the process of the invention is at least 1%, 2%, 3%, 4%, 5%, 6%, 7%, 8%, 9%, 10% or even more of the initial L-lactate content. Each possibility represents a separate embodiment. For example, for an initial enantiomeric mixture containing 90% L-lactate and 10% D-lactate, a 10% enrichment results in magnesium lactate salt containing 99% L-lactate and 1% D-lactate. Within the scope of the present invention is the reduction in D-lactate content by the process disclosed herein by at least 10%, 15%, 20%, 25%, 30%, 35%, 40%, 45%, 50%, 55%, 60%, 65%, 70%, 75%. 80%, 85%, 90%, 95%, or even 100% of the initial D-lactate content. Each possibility represents a separate embodiment. For example, for an initial enantiomeric mixture containing 90% L-lactate and 10% D-lactate, a 50% reduction in D-lactate content results in magnesium lactate salt containing 95% L-lactate and 5% D-lactate. The obtained magnesium L-lactate crystals according to the principles of the present invention comprise less than 3% magnesium D-lactate, less than 2% magnesium D-lactate, less than 1.5% magnesium D-lactate, or less than 1% magnesium D-lactate. Each possibility represents a separate embodiment.

An additional advantage stemming from the processes of the present invention is the enrichment in purity of magnesium L-lactate. Typically, the initial purity of the lactate in the decomposed organic waste is low. There are many non-lactate impurities which are present in the organic waste and are carried over to the decomposition product thereof. Advantageously, the present invention provides a purity of at least 80% of the crude magnesium L-lactate formed directly using the process disclosed herein. Additional enrichment in purity can be affected by washing, crystallization or recrystallization processes to yield highly pure magnesium L-lactate salt.

According to the principles of the present invention, the decomposed organic waste used in the process disclosed herein is a decomposition product of any lactic acid-containing waste such as, but not limited to, polylactic acid polymer which was subjected to hydrolysis using an alkaline compound e.g. sodium hydroxide. According to additional embodiments, the decomposed organic waste used in the process disclosed herein is obtained from lactic acid fermentation of fermentable carbohydrates such as, but not limited to, those derived from organic waste. The organic waste feedstocks within the scope of the present invention can originate from any waste source including, but not limited to, food waste, municipal food waste, residential food waste, agricultural waste, industrial food waste from food processing facilities, commercial food waste (from hospitals, restaurants, shopping centers, airports etc.), and a mixture or combination thereof. Each possibility represents a separate embodiment. The organic waste can additionally originate from residues ranging from animal and human excreta, vegetable and fruit residues, plants, cooked food, protein residues, slaughter waste, and combinations thereof. Each possibility represents a separate embodiment. Industrial organic food waste may include factory waste such as byproducts, factory rejects (e.g. expired products, defective products), market returns or trimmings of inedible food portions (such as skin, fat, crusts and peels). Each possibility represents a separate embodiment. Commercial organic food waste may include waste from shopping malls, restaurants, supermarkets, etc. Each possibility represents a separate embodiment.

According to certain aspects and embodiments, the organic waste comprises monosaccharides or disaccharides obtained as byproducts of sugar production from beet sugar or cane sugar such as, but not limited to, production of fructose, molasses, or high fructose corn syrup (HFCS). According to other aspects and embodiments, the organic waste comprises starches and starch derivatives such as refined glucose syrups originating from the hydrolysis of starch, which starches may be maize starch, tapioca starch, wheat starch, potato starch, and the like. Each possibility represents a separate embodiment. The organic waste may further be derived from byproducts of wine or beer production such as, but not limited to, yeast autolysates and hydrolysates as well as from plant protein hydrolysates, animal protein hydrolysates, and soluble byproducts from steeping wheat or maize. Paper sludge hydrolysate obtained by hydrolyzing paper sludge with cellulolytic enzymes may also be used as well as dairy byproducts generated during cheese production and dairy beverages production of milk-based beverages including e.g. lactose-free beverages.

According to various aspects and embodiments, the decomposed organic waste comprises a fermentation broth obtained from a fermentation process of a carbohydrate source. When using non-homogenous feedstocks, the decomposed organic waste or fermentation broth typically comprises insoluble organic-based impurities such as, but not limited to, microorganisms (e.g. lactic acid producing microorganisms including e.g. yeasts, bacteria and fungi), fats and oils, lipids, aggregated proteins, bone fragments, hair, precipitated salts, cell debris, fibers (e.g. fruit and/or vegetables peels), and residual unprocessed waste (e.g. food shells, seeds, food insoluble particles and debris, etc.). Each possibility represents a separate embodiment. Non-limiting examples of insoluble inorganic-based impurities include plastics, glass, residues from food packaging, sand, and combinations thereof. Each possibility represents a separate embodiment.

Although not necessary, the decomposed organic waste can further be pre-treated prior to employing the processes of the present invention. Suitable pre-treatment includes, but is not limited to, filtration, ultrafiltration, nanofiltration, reverse osmosis (RO) filtration, solvent extraction, repulsive extraction, salt precipitation, crystallization, distillation, evaporation, electrodialysis, and diverse types of chromatography (such as adsorption or ion exchange). Each possibility represents a separate embodiment.

The decomposed waste may contain various concentrations of D- and L-lactate. The processes of the present invention advantageously provide high purity magnesium L-lactate even when the initial concentration of D- and L-lactate monomers is as low as 10%. Typically, the initial concentration of D- and L-lactate monomers is in the range of about 20% to about 50%, including each value within the specified range. The ratio of D- and L-lactate monomers in the decomposed waste may vary according to the endogenous D-lactate content as well as racemic lactic acid formation which occurs during decomposition. Typically, ratios of D- to L-lactate monomers include, but are not limited, 1:99, 2:98, 3:97, 4:96, 5:95, 6:94, 7:93, 8:92, 9:91, 10:90, 11:89, 12:88, 13:87, 14:86, 15:85, 16:84, 17:83, 18:82, 19:81, or 20:80. Each possibility represents a separate embodiment.

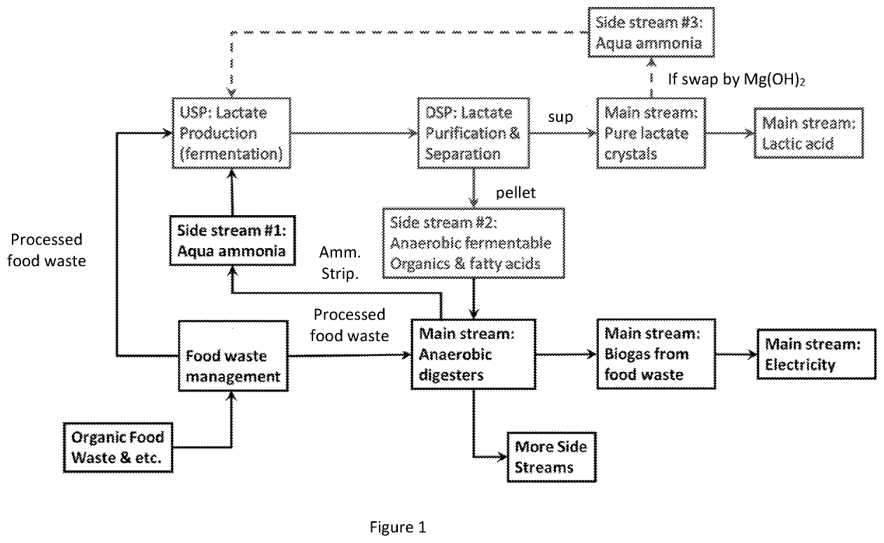

Referring now to the drawings, FIG. 1 illustrates an operation scheme for the production of magnesium L-lactate according to certain embodiments of the present invention. Lactic acid fermentation is performed. Organic waste such as municipal waste, food waste and agricultural waste serves as the substrate for L-lactic acid fermentation by L-lactic acid-producing microorganisms e.g. Bacillus coagulans. Due to the formation of L-lactic acid, endogenous lowering of the pH occurs. Thus, the fermentation process is carried out in the presence of an alkaline substance to adjust the pH during fermentation. The alkaline substance neutralizes the pH resulting in the formation of L-lactate ions and counterions. Decomposition of the organic waste typically involves an alkaline substance selected from sodium hydroxide, potassium hydroxide or ammonium hydroxide, to thereby generate sodium, potassium or ammonium counterions, respectively, in the decomposed waste. Thus, the use of sodium hydroxide results in the formation of sodium lactate with sodium being the counterion, and the use of ammonium hydroxide results in the formation of ammonium lactate with ammonium being the counterion. Advantageously, the alkaline substance added to the lactic acid fermentation is ammonia water or aqua ammonia. The use of ammonia water enables the additional recycling of waste that remains after the fermentation process. This mode of operation is termed a “counter current” in which the source of the alkaline compound used in one batch production of lactic acid is obtained from a previous batch production of lactic acid. In particular, while the main stream of lactic acid production continues to downstream processing of lactate purification and separation, the biomass derived from the fermentation can be reused as a side stream (#2: anaerobic fermentable organics & fatty acids). In this side stream, anaerobic digestion is performed to produce, as a main product, biogas (primarily methane). When the biomass that remained after anaerobic digestion is concentrated, it can be used to generate ammonia water e.g. by gas stripping. The ammonia water can then be used in a subsequent fermentation process to adjust the pH. This mode of operation is cost-effective as it enables the reduction in costs of expensive alkaline compounds to neutralize the pH during fermentation. As an additional ammonia water source, the scheme demonstrates a step of ion exchange or swap of the lactate counterion (NH₄⁺) in the fermentation broth by using e.g. Mg(OH)₂ thereby providing additional ammonia water that can be circulated back to a subsequent fermentation batch. Additional use for the biomass that remains following fermentation is for supplementing recycling streams that have low nutrients content such as, but not limited to, paper waste hydrolysates, yeast lysate, and dairy water.

According to certain aspects and embodiments, the decomposed organic waste may optionally be neutralized using an acid or a base as is known in the art. Typically, neutralization is performed to a pH of about 6.5-7.5 including any value therebetween. It will be appreciated by one skilled in the art that when the pH of the decomposed organic waste is basic, neutralization is performed by the addition of an acid. Suitable acids include, but are not limited to, hydrochloric acid, hydrobromic acid, nitric acid, phosphoric acid, sulfuric acid, and combinations thereof. Each possibility represents a separate embodiment. Alternatively, when the pH of the decomposed organic waste is acidic, neutralization is performed by the addition of a base. Suitable bases include, but are not limited to, sodium, potassium, or ammonium hydroxide, and combinations thereof. Each possibility represents a separate embodiment.

In case the decomposed organic waste contains particles of PLA waste which have failed to be hydrolyzed or non-lactic acid-containing impurities, they may be separated from the decomposed organic waste, for example by solid-liquid separation techniques such as filtration or decantation. Each possibility represents a separate embodiment.

According to the principles of the present invention, ion swap is then performed by the addition of a magnesium salt to result in the precipitation of magnesium L-lactate salt with enriched overall purity, and enantiomeric purity. It is to be understood that the ion swap of the present invention can be complete, i.e. the counterion derived from the alkaline substance is other than magnesium, or partial, i.e. two or more alkaline substances are used, one of which contains magnesium ions and the other contains cations other than magnesium. The magnesium salt used for the ion swap can be added in solid form or as an aqueous solution. Each possibility represents a separate embodiment. When magnesium salt is added as an aqueous solution, typically the aqueous solution contains magnesium ions at a concentration ranging from about 50 to about 500 g/L, including each value within the specified range. Exemplary magnesium ion concentrations include, but are not limited to, from about 75 g/L to about 400 g/L, from about 100 g/L to about 300 g/L, or from about 150 g/L to about 250 g/L, including each value within the specified ranges. Each possibility represents a separate embodiment. In some embodiments, the magnesium salt is gradually added while mixing.

In certain aspects and embodiments, the magnesium salt is derived from an acidification, methylation or acetylation of a lactate salt from a previous batch. In accordance with these embodiments, the salt is a recycled salt thereby conferring an additional advantage of cost savings. Typically, the processes of the present invention produce a magnesium lactate salt as the end product. However, in order to obtain polylactic acid, the lactate salt may be acidified for polymerization using e.g. hydrochloric acid, hydrobromic acid, nitric acid, phosphoric acid, sulfuric acid, and a mixture or combination thereof. Each possibility represents a separate embodiment. Following acid addition, the magnesium ions can precipitate together with the anion of the acid to form a magnesium salt which can be used for a subsequent ion swap according to the principles of the present invention. Where the lactate salt product undergoes methylation or acetylation, these processes are also typically performed in the presence of an acid thereby leading to the precipitation of a magnesium salt that can be used in a subsequent ion swap process.

Magnesium salts within the scope of the present invention include, but are not limited to, magnesium chloride, magnesium carbonate, magnesium sulfate, magnesium phosphate, magnesium hydroxide and the like. Each possibility represents a separate embodiment. The magnesium salts, according to the principles of the present invention, include any hydrate or anhydrous forms such as, but not limited to, MgCl₂·xH₂O where x=0-6. MgCO₃·xH₂O where x=0, 1, 2, 3, or 5, MgSO₄·xH₂O where x=0-11, Mg₃(PO₄)₂·xH₂O where x=0, 5, 8, or 22, MgHPO₄·xH₂O where x=0 or 3, Mg(H₂PO₄)₂·xH₂O where x=0, 2 or 4, Mg(OH)₂ and the like. Each possibility represents a separate embodiment. In one embodiment, magnesium sulfate (e.g. magnesium sulfate heptahydrate) is added.

In some aspects and embodiments, the magnesium salt is added in stochiometric amounts. In other aspects and embodiments, the magnesium salt is added in excess. Up to 20% excess of the magnesium salt can be added according to the principles of the present invention.

Within the scope of the present invention is the addition of magnesium salt at room temperatures or at elevated temperatures. Each possibility represents a separate embodiment. Suitable temperatures include a range of 20° C. to 80° C., for example about 20° C., 25° C., 30° C., 35° C., 40° C., 45° C., 50° C., 55° C., 60° C., 65° C., 70° C., 75° C., or 80° C. Each possibility represents a separate embodiment.

The thus obtained magnesium L-lactate salt may further be subjected to downstream purification processes. One simple purification that has also surprisingly been found to improve the enantiomeric purity of magnesium L-lactate is washing the crude magnesium L-lactate salt, for example using purified water. Within the scope of the present invention are additional purification steps, for example, crystallization, recrystallization, partitioning, silica gel chromatography, preparative HPLC, and combinations thereof. Each possibility represents a separate embodiment. A re-acidification step may also be carried out in order to obtain crude L-lactic acid, followed by purification steps to obtain a purified L-lactic acid. Re-acidification can be performed as is known in the art, for example by using at least one of hydrochloric acid, hydrobromic acid, nitric acid, phosphoric acid, sulfuric acid, and combinations thereof. Each possibility represents a separate embodiment.

The purification processes may include extraction, electrodialysis, adsorption, ion-exchange, crystallization, and combinations of these methods. Several methods are reviewed, for example, in Ghaffar et al. (2014) Journal of Radiation Research and Applied Sciences, 7 (2): 222-229; and López-Garzón et al. (2014) Biotechnol Adv., 32 (5): 873-904. Alternatively, recovery and conversion of lactic acid to lactide in a single step may be used (Dusselier et al. (2015) Science, 349 (6243): 78-80).

A particular downstream purification process for purifying magnesium lactate via crystallization is described in a co-pending patent application, WO 2020/110108, assigned to the Applicant of the present invention. The purification process comprises the following steps:
providing a clarified solution containing magnesium lactate at a temperature between 45° C. to 75° C.;
concentrating the solution to a concentration of 150-220 g/L of lactate;
performing at least one cooling crystallization of the concentrated solution to obtain magnesium lactate crystals; and
collecting the magnesium lactate crystals obtained.

In some embodiments, the solution is provided at a temperature between 55° C. to 65° C., including each value within the specified range.

The concentration of the solution may be performed by evaporation, nanofiltration, reverse osmosis, or combinations thereof. Each possibility represents a separate embodiment. In some embodiments, the solution is concentrated to a concentration of 160-220 g/L of lactate, for example, 170-220 g/L of lactate, or 180-220 g/L of lactate, including each value within the specified ranges.

The at least one cooling crystallization may begin at a first temperature in the range of 50 to 75° C., including each value within the specified range. In some embodiments, the at least one cooling crystallization begins at a first temperature in the range of 50 to 70° C. including each value within the specified range. In additional embodiments, the at least one cooling crystallization begins at a first temperature in the range of 50 to 65° C., including each value within the specified range.

The at least one cooling crystallization step may end at a second temperature in the range of 10 to 1° C., including each value within the specified range. In some embodiments, the at least one cooling crystallization ends at a second temperature in the range of 6 to 2° C., including each value within the specified range.

The cooling rate of the at least one cooling crystallization may be in the range of 10 to 0.5° C./h, including each value within the specified range. In some embodiments, the cooling rate is in the range of 5 to 1° C./h, including each value within the specified range.

Before the cooling crystallization, the pH of the concentrated mixture may be adjusted to be in the range of 6 to 7, including each value therebetween.

The obtained magnesium lactate crystals may be separated from the remaining liquid by microfiltration or nanofiltration. The remaining liquid may undergo concentration, followed by at least one additional cooling crystallization, in order to obtain additional magnesium lactate crystals. Following their separation from the liquid, the magnesium lactate crystals may be washed with an aqueous solution or with an organic solvent such as ethanol or acetone and purified. Further processing of the magnesium lactate crystals may include at least one of extraction, microfiltration, nanofiltration, active carbon treatment, drying and grinding. Each possibility represents a separate embodiment.

While the processes disclosed herein are primarily contemplated for enriching L-lactate enantiomer from an enantiomeric mixture comprising D- and L-lactate derived from decomposed organic waste, enrichment of the D-lactate enantiomer is also contemplated by the present invention.

Thus, according to certain aspects and embodiments, the present invention provides a process for enriching D-lactate enantiomer from an enantiomeric mixture derived from decomposed organic waste, the process comprising the steps of:
(a) obtaining decomposed organic waste comprising an enantiomeric mixture of D- and L-lactate and a counterion other than magnesium;
(b) optionally performing at least one of neutralizing the D- and L-lactate and removing solid particles from the decomposed organic waste; and
(c) adding a magnesium salt to the enantiomeric mixture of step (a) or (b) to thereby precipitate magnesium D-lactate salt with enriched enantiomeric purity.

According to other aspects and embodiments, the present invention provides a process for producing magnesium D-lactate salt from decomposed organic waste in high purity, the process comprising the steps of:
(a) obtaining decomposed organic waste comprising D-lactate and a counterion other than magnesium;
(b) optionally performing at least one of neutralizing the D-lactate and removing solid particles from the decomposed organic waste; and
(c) adding a magnesium salt to the decomposed organic waste of step (a) or (b) to thereby precipitate magnesium D-lactate salt in high purity.

As used herein and in the appended claims, the term “about” refers to ±10%.

As used herein and in the appended claims, the singular forms “a”, “an”, and “the” include plural references unless the context clearly dictates otherwise. Thus, for example, reference to “a counterion” includes a plurality of such counterions unless the context clearly dictates otherwise. It should be noted that the term “and” or the term “or” are generally employed in their sense including “and/or” unless the context clearly dictates otherwise.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Alkaline Thermohydrolysis of PLA Pellets Using Sodium Hydroxide

50 g of PLA pellets (Ingeo™ Biopolymer 4032D, Nature Works LLC.) were added to a 250 ml three-necked flask equipped with a condenser and a thermometer. 150 ml of NaOH 5M were added, and the flask was heated to 80° C. (pH measured was 13.5).

After 3.5 hours of rapid degradation, the lactate concentration reached 320 g/L with only minor further increase in lactate concentration over time. After 21.5 hours, the lactate concentration stopped increasing (final concentration of 340 g/L) and the reaction was cooled to room temperature.

PLA residues were filtered using a sintered glass funnel to result in a clear solution. The final pH measured was 12.9 which is suitable for additional PLA degradation.

The solution was neutralized with concentrated H₂SO₄, then 280 ml of magnesium sulfate heptahydrate solution (300 g/L) were added dropwise while stirring. The MgLa₂·2H₂O precipitate that formed was filtered using a sintered glass funnel, washed with acetone, and dried at 80° C. to a final weight of 64 gr. The filtrate was added dropwise into 500 ml of acetone while stirring, and then stirred for another hour. The precipitate that formed was filtered using a sintered glass funnel, washed with acetone, and dried at 80° C. Yield: 74%.

### Example 2

### Recycling of PLA Through MgLa₂·2H₂O Precipitation

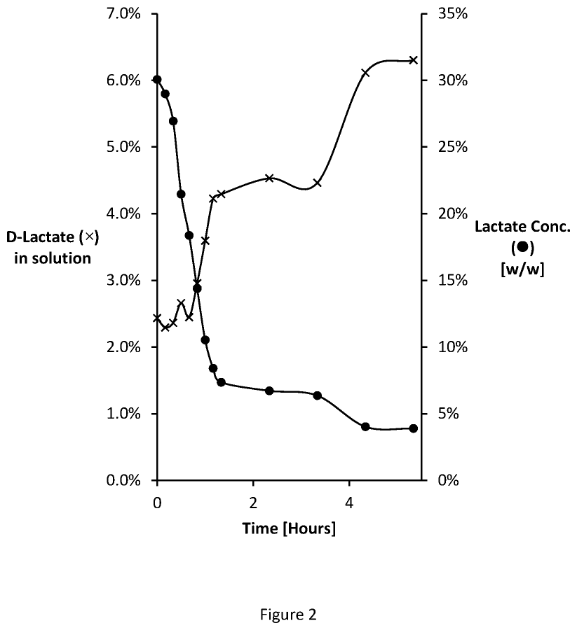

In an appropriate stainless-steel vessel, PLA (2 kg) obtained from 4032D PLA pellets or recycled from cafeteria waste was added to a stirred 5M sodium hydroxide solution (4 L) and heated to 90° C. until the lactate concentration reached about 30%. The solution was allowed to cool down to RT, filtered-off (0.45 μm cutoff) from undissolved leftovers and the pH of the filtrate was neutralized to 6.5-7.0 using concentrated sulfuric acid. Then, substantial portion (>85%) of the lactate was recuperated as solid MgLa₂·2H₂O by a swap reaction at 20-80° C. with 0-20% excess of MgSO₄·xH₂O (x=0-11) which was added to the mixed solution portion-wise. The precipitated solid was then filtered off and washed once with RT water to typically afford crude MgLa₂·2H₂O in >80% purity. Rinsing the crude magnesium lactate once with water normally improved purity to >95%. This swap procedure was also applied to a commercial NaLa solution containing 2.4% of D-lactate. The concentration of the D-lactate enantiomer was measured using chiral HPLC separation. Surprisingly, the protocol yielded a decrease in the D- to L-lactate ratios. In another experiment, MgLa₂ was extracted from a 30% w/w sodium lactate solution obtained from degradation of Natureworks 4032D PLA pellets according to the aforementioned protocol. Both the lactate concentration and % of D-lactate were measured separately using HPLC. As can be seen in FIG. 2, the overall lactate concentration in solution dropped from 30 to 4% while the % D-lactate in solution (of the total lactate content) increased from 2.4 to 6.3%. This enrichment of % D-lactate in solution provides improved enantiomeric purity of the precipitated MgLa₂ that contains less of the D enantiomer than the initial D enantiomer content. The results are summarized in Tables 1A-1B.

TABLE 1A
General extraction yield and purity of the obtained MgLa₂:

| | | NaLa from | NaLa from |
|---|---|---|---|
| | Commercial | degradation of | degradation of |
| | NaLa solution | PLA 4032D | cafeteria waste |
| Lactate | 85.6% | 89.7% | 91.8% |
| recuperation | | | |
| yield as MgLa₂ | | | |
| Purity of | 92.1% | 90.4% | 83.2% |
| crude MgLa₂ | | | |
| precipitate | | | |
| Purity of | 98.5% | 94.7% | 96.2% |
| washed MgLa₂ | | | |
| precipitate | | | |

TABLE 1B
Enantiomeric purity:

| | % D-Lactate in | | |
|---|---|---|---|
| | | NaLa from | NaLa from |
| | Commercial | degradation of | degradation of |
| | NaLa | PLA 4032D | cafeteria waste |
| 30% NaLa solution | 2.7% | 2.4% | 4.9% |
| at t = 0 of swap | | | |
| Solution at the end | 3.5% | 6.3% | 7.0% |
| of the swap | (Δ = +0.8%) | (Δ = +3.9%) | (Δ = +2.1%) |
| Filtered MgLa₂ | 2.4% | 1.3% | 3.8% |
| precipitate | | | |
| Washed MgLa₂ | 1.6% | 1.3% | 1.3% |

Thus, the results demonstrate that the process of the present invention enhances the enantiomeric purity of lactate by selectively precipitating magnesium L-lactate crystals thus resulting in increased D-lactate concentrations in solution. The magnesium lactate obtained by this process is particularly suitable for re-polymerization of the recycled lactate to PLA.

### Example 3

### Recycling of Organic Waste in a Lactic Acid Fermentation Process

Mixed food waste is decomposed by lactic acid fermentation in the presence of NaOH or NH₄OH as a pH adjusting alkaline compound. The thus obtained fermentation broth contains lactate ions and sodium or ammonium counterions. The broth is allowed to cool to RT followed by filtration to remove undissolved material. Then, lactate is recuperated as solid MgLa₂·2H₂O by a swap reaction at 20-80° C. with 0-20% excess of MgSO₄·xH₂O (x=0-11) which is added to the mixed solution. The lactate concentration and % of D-lactate are measured separately using HPLC. The precipitated solid is then filtered off and washed with water to typically afford crude MgLa₂·2H₂O. Optionally, the crude magnesium lactate is rinsed with RT water and subject to subsequent recrystallization.

### Example 4

### L-lactate Enrichment

Ammonium lactate solution, either purchased commercially or sourced from a food waste fermentation broth, was brought to an initial concentration of 30% lactate. About 250 g of the solution was transferred to a 1 L three-necked round bottom flask equipped with a condenser and a mechanical stirrer. The solution was heated to 70° C. and the initial pH of 5.5 was adjusted to 7.2 by the addition of ammonium hydroxide. 1.1 molar equiv. of MgSO₄ was added in 8 equal portions during 1.5 h, resulting in precipitation of magnesium lactate. The mixture was stirred for another 2 h and then filtered using a P3 sintered glass funnel. The filtered precipitate was washed with 1 weight eq. of cold water and dried at 70° C. The lactate concentration and % of D-lactate were measured separately using HPLC. The results are summarized in Tables 2A-2B.

TABLE 2A
General extraction yield and purity of the obtained MgLa₂:

| | Commercial | NH₄La from food |
|---|---|---|
| | NH₄La | waste fermentation |
| Lactate recuperation | 67.0% | 78.0% |
| yield as MgLa₂ | | |
| Purity of crude MgLa₂ | 89.6% | 79.5% |
| precipitate | | |
| Purity of washed MgLa₂ | 98.2% | 93.9% |
| precipitate | | |

TABLE 2B
Enantiomeric purity:

% D-Lactate in

| | Commercial | NH₄La from food |
|---|---|---|
| | NH₄La | waste fermentation |
| 30% NH₄La solution | 5.6% | 5.2% |
| at t = 0 of swap | | |
| Solution at the end | 12.6% | 12.8% |
| of the swap | | |
| Filtered MgLa₂ | 2.5% | 3.0% |
| precipitate | | |
| Washed MgLa₂ | 2.0% | 2.3% |

The results demonstrate that the process of the present invention enhances the enantiomeric purity of the L-lactate salt. Advantageously, more than 50% reduction in D-lactate content was achieved even when utilizing NH₄ La derived from food waste fermentation which contains significant amounts of impurities.

### Example 5

### L-lactate Enrichment by an Ion Swap Followed by Re-Crystallization

Ammonium lactate solution, sourced from a food waste fermentation broth, was brought to a starting concentration of 29% lactate. About 250 g of the solution were transferred to a 1 L three-necked round bottom flask equipped with a condenser and a mechanical stirrer. The solution was heated to 70° C. and the initial pH of 5.7 was adjusted to 7.3 by the addition of ammonium hydroxide. 1.1 molar equiv. of MgSO₄ was added in 8 equal portions during 1.5h, resulting in precipitation of magnesium lactate. The mixture was stirred for another 4h and then filtered using a P3 sintered glass funnel. The filtered precipitate was washed with 1 weight eq. of cold water and dried at 70° C. The dry magnesium lactate precipitate was then re-dissolved in water to a concentration of 8.4% lactate. 5% w/w of active carbon were added, and the solution was stirred overnight. The active carbon was filtered off and the clarified solution was transferred to a 0.5 L reactor pre-heated to 70° C. and stirred at 300 RPM. The solution was concentrated in vacuo, removing 75% of the water, resulting in crystallization of the magnesium lactate. The crystals were then harvested and filtered using a sintered glass funnel. The obtained crystals were washed with cold water and dried at 70° C. The results are summarized in Tables 3A-3D.

TABLE 3A
Lactate concentration and D-lactate content in solution after swap:

| | | % D-lactate of total |
|---|---|---|
| | Lactate conc. [%] | lactate |
| Ammonium lactate solution | 29.2% | 2.9% |
| Swap filtrate | 6.1% | 8.0% |
| Wash filtrate | 5.6% | 3.2% |

TABLE 3B
Purity, D-lactate content, and yield of the precipitate after swap:

| | | % D-lactate of total | |
|---|---|---|---|
| | Purity [%] | lactate | % Yield |
| magnesium lactate | 83.4% | 1.7% | 75% |
| precipitate | | | |
| washed magnesium | 96.8% | 1.4% | 64% |
| lactate precipitate | | | |

TABLE 3C
Lactate concentration and D-lactate content
in solution after re-crystallization:

| | | % D-lactate of total |
|---|---|---|
| | Lactate conc. [%] | lactate |
| Re-crystallization solution | 8.4% | 1.4% |
| Mother liquor | 7.7% | 5.1% |
| Wash filtrate | 3.6% | 2.3% |

TABLE 3D
Purity, D-lactate content, and yield of
the precipitate after re-crystallization:

| | | % D-lactate of total | |
|---|---|---|---|
| | Purity [%] | lactate | % Yield |
| magnesium lactate | 99.2% | 1.2% | 81% |
| crystals | | | |
| washed magnesium | 99.5% | 0.8% | 71% |
| lactate crystals | | | |

The magnesium lactate crystals following re-crystallization contain less than 1% D-lactate and are therefore particularly suitable for reuse in the formation of new polylactic acid.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the spirit and scope of the present invention as described by the claims, which follow.

1-38. (canceled)

39. A process for enriching L-lactate enantiomer from an enantiomeric mixture derived from decomposed organic waste, the process comprising the steps of:
(a) obtaining decomposed organic waste comprising an enantiomeric mixture of D- and L-lactate and a counterion other than magnesium;
(b) optionally performing at least one of neutralizing the D- and L-lactate and removing solid particles from the decomposed organic waste; and
(c) adding a magnesium salt to the enantiomeric mixture of step (a) or (b) to thereby precipitate magnesium L-lactate salt with enriched enantiomeric purity.

40. The process of claim 39 comprising enrichment of L-lactate enantiomer by 1% or more.

41. The process of claim 39, wherein the decomposed organic waste is obtained from a lactic acid fermentation process or wherein the decomposed organic waste is obtained from hydrolysis of polylactic acid polymer.

42. The process of claim 39, wherein the decomposed organic waste is pre-treated prior to step (a) to remove non-lactic acid-containing impurities.

43. The process of claim 39, wherein the enantiomeric mixture comprises 20% D-lactate or less.

44. The process of claim 39, wherein the counterion is selected from the group consisting of sodium, potassium and ammonium.

45. The process of claim 39, wherein step (b) comprising neutralizing the D- and L-lactate is performed.

46. The process of claim 45, wherein neutralizing the D- and L-lactate comprises the addition of an acid selected from hydrochloric acid, hydrobromic acid, nitric acid, phosphoric acid, sulfuric acid, and combinations thereof, or wherein neutralizing the D- and L-lactate comprises the addition of a base selected from sodium, potassium, or ammonium hydroxide, and combinations thereof.

47. The process of claim 39, wherein step (b) comprising removing solid particles from the decomposed organic waste is performed and removal of solid particles comprises solid-liquid separation.

48. The process of claim 39, wherein step (c) is performed at elevated temperatures in the range of 20° C. to 80° C.

49. The process of claim 39, wherein the magnesium salt in step (c) is added in solid form: or wherein the magnesium salt in step (c) is added as an aqueous solution; or wherein the magnesium salt in step (c) is added in excess of up to 20%; or wherein the magnesium salt in step (c) is magnesium sulfate.

50. The process of claim 39, wherein the obtained magnesium L-lactate salt is separated by filtration or centrifugation; or wherein the obtained magnesium L-lactate salt is subjected to subsequent purification.

51. The process of claim 50, wherein subsequent purification comprises at least one of crystallization, recrystallization, partitioning, silica gel chromatography, and preparative HPLC: or wherein subsequent purification comprises washing the obtained magnesium L-lactate salt with purified water.

52. The process of claim 39, wherein the obtained magnesium L-lactate salt comprises less than 3% magnesium D-lactate: or wherein the obtained magnesium L-lactate salt is crystalline magnesium L-lactate dihydrate; or wherein the obtained magnesium L-lactate is acidified to form L-lactic acid by at least one of hydrochloric acid, hydrobromic acid, nitric acid, phosphoric acid, sulfuric acid, and combinations thereof and used for subsequent polylactic acid formation.

53. The process of claim 39, further comprising enriching the purity of L-lactate salt from decomposed organic waste.

54. A process for producing magnesium L-lactate salt from decomposed organic waste in high or enriched purity, the process comprising the steps of:
(a) obtaining decomposed organic waste comprising L-lactate and a counterion other than magnesium:
(b) optionally performing at least one of neutralizing the L-lactate and removing solid particles from the decomposed organic waste; and
(c) adding a magnesium salt to the decomposed organic waste of step (a) or (b) to thereby precipitate magnesium L-lactate salt in high or enriched purity.

55. A process for producing magnesium L-lactate salt from decomposed organic waste, the process comprising the steps of:
(a) decomposing organic waste by performing at least one of organic waste fermentation using a lactic acid-producing microorganism and PLA hydrolysis in the presence of an alkaline compound to obtain decomposed organic waste comprising L-lactate and a counterion other than magnesium:
(b) optionally performing at least one of neutralizing the L-lactate and removing solid particles from the decomposed organic waste; and
(c) adding a magnesium salt to the decomposed organic waste of step (a) or (b) to thereby precipitate magnesium L-lactate salt.

56. The process of claim 55, wherein the alkaline compound comprises at least one of NaOH, KOH, NH₄OH, Ca(OH)₂, and a mixture or combination thereof; or wherein the alkaline compound comprises a combination of Mg(OH)₂ and/or MgCO₃ and at least one of NaOH, KOH, NH₄OH, Ca(OH)₂, and a mixture or combination thereof; or wherein the alkaline compound comprises NH₄OH derived from anaerobic digestion of a solid biomass obtained from a previous batch of lactic acid fermentation.

57. The process of claim 56, wherein the NH₄OH is obtained by gas stripping.

58. The process of claim 55, wherein the magnesium salt in step (c) is derived from acidification, methylation or acetylation of magnesium L-lactate of a previous batch of lactic acid fermentation; or wherein the magnesium salt in step (c) is derived from acidification, methylation or acetylation of magnesium L-lactate of a previous batch of PLA hydrolysis.
